(19) United States
(12) Patent Application Publication
(10) Pub. No.: US 2004/0132062 A1
(43) Pub. Date: Jul. 8, 2004

(54) Genetic diagnosis of depression

(75) Inventors: Boris Tabakoff, Denver, CO (US); Larry Martinez, Edmonds, WA (US)

Correspondence Address: Christine A. Lekutis, MEDLEN & CARROLL, LLP, Suite 350, 101 Howard Street, San Francisco, CA, 94105, US

(73) Assignee: The Regents of the University of Colorado, a body corporate, Boulder, CO

(21) Appl. No.: 10/670,452

(22) Filed: Sep. 24, 2003

Related U.S. Application Data
(60) Provisional application No. 60/413,318, filed on Sep. 24, 2002.

(51) Int. Cl. C12Q 1/68; C12P 19/34

(52) U.S. Cl. 435/6; 435/91.2

(57) ABSTRACT

The present invention relates to compositions and methods for determining whether an individual is predisposed to depression. In particular, the present invention provides a genetic marker useful alone or in combination with other genetic markers for the diagnosis, characterization and treatment of depression

This application claims priority to U.S. Patent Application No. 60/413,318, filed on Sep. 24, 2002.

### FIELD OF THE INVENTION

The present invention relates to compositions and methods for determining whether an individual is predisposed to depression. In particular, the present invention provides a genetic marker useful for the diagnosis, characterization and treatment of depression.

### BACKGROUND OF THE INVENTION

Major depression is a persistent, disabling mood disorder characterized by sadness, loss of interest, and/or irritability, in the absence of mood-incongruent psychosis or mania (See, e.g., Hyman and Rudorfer, “Depressive and Bipolar Mood Disorders,” in Scientific American Medicine, volume 3, 2000; and Diagnostic and Statistical Manual of Mental Disorders, 4th edition, American Psychiatric Association, Washington D.C., 1994). Symptoms of major depression include: appetite or weight fluctuations, sleep disturbances, agitation, fatigue, inappropriate self-reproach or guilt, poor concentration or inability to make decisions, and suicidal thoughts. Importantly, patients suffering from major depression experience these symptoms as a result of their mood disorder, as opposed to as a result of physical illness, medication, substance abuse or normal bereavement.

The presence of unipolar depression in the United States is approximately 10%, with women experiencing depression twice as frequently as men (Regier et al., Arch Gen Psychiatry 45:977, 1988). In the Unites States, major depression ranks first among all causes of disability and second after heart disease as a cause of healthy years lost to premature morbidity and mortality (Murray and Lopez, Lancet, 349:1436, 1997). This problem is exacerbated by the fact that depression is commonly misdiagnosed and/or inadequately treated (Hirschfeld et al., JAMA, 277:333, 1997).

Many depressed patients receive benefit from antidepressant drugs and/or psychotherapy. Antidepressant drugs are currently classified according to their chemical structure and method of action. The three main categories of antidepressants include the tricyclic antidepressants, second generation antidepressants (e.g., neurotransmitter reuptake inhibitors and neurotransmitter agonists) and monoamine oxidase inhibitors. The efficacy of these types of medications, have implicated the monoamine systems, utilizing the neurotransmitters serotonin, norepinephrine, and dopamine, in the pathophysiology of depression. However, it is unclear whether biogenic amine deficits themselves cause depression or whether defects in their targets are responsible for precipitating mood disorders.

Strikingly, the prevalence of major depression in first-degree relatives of depressed patients is nearly three times that of the individuals who do not have a family history of depression (Sullivan et al., Am J Psychiatry, 157:1552-1562, 2000). In fact, the most well-validated risk factor for depression is family history (Weisman et al., Arch Gen Psychiatry, 54:932, 1997). Twin and adoption studies provide further support for the theory that there is a genetic component to depression (Sullivan et al., supra 2000; and Wender et al., Arch Gen Psychiatry, 43:923, 1986). The most well studied locus associated with depression and other psychiatric disorders is the serotonin (e.g., 5-hydroxytryptamine) transporter (5-HTT, See, e.g., Ogilvie et al., Lancet 347:731-733, 1996).

However, hereditary studies of depression suggest that multiple genetic loci, as well as environmental factors, predispose individuals to developing depression. Thus, there remains a need in the art for the identification of additional genes that play a role in major depression. In particular, the molecular definition of polymorphisms at depression loci will prove useful for accurate diagnosis of depression. Identification of depression susceptibility alleles is also contemplated to provide tools for the screening of new antidepressants and for the selection of appropriate medications.

### SUMMARY OF THE INVENTION

The present invention relates to compositions and methods for determining whether an individual is predisposed to depression. In particular, the present invention provides a genetic marker useful alone or in combination with other genetic markers for the diagnosis, characterization and treatment of depression.

In particular, the present invention provides methods of identifying individuals predisposed to major depressive disorder comprising: providing a nucleic acid from a human subject; wherein the nucleic acid comprises an adenylyl cyclase type 7 allele; detecting the presence of at least one polymorphism within the adenylyl cyclase type 7 allele; and correlating the presence of the at least one polymorphism with a predisposition to major depressive disorder. In some preferred embodiments, the at least one polymorphism is a repeat polymorphism, while in some particularly preferred embodiments, the repeat polymorphism is an [AACA]₇ repeat in the 3′ untranslated region of the adenylyl cyclase type 7 allele. In some preferred embodiments, the subject is Caucasian. In particularly preferred embodiments, the subject is female. In other preferred embodiments, the subject is alcohol-dependent. The present invention provides embodiments wherein the detecting step is accomplished using at least one technique selected from the group consisting of polymerase chain reaction, heteroduplex analysis, single stand conformational polymorphism analysis, ligase chain reaction, comparative genome hybridization, Southern blotting and sequencing. In some embodiments, the nucleic acid from the subject is derived from a sample selected from the group consisting of buccal cells, biopsy material and blood. In some preferred embodiments, the methods further comprise providing a diagnosis to the subject based on the presence or absence of the polymorphism. In particularly preferred embodiments, the diagnosis differentiates major depressive disorder from other forms of mental illness. In some embodiments, the other forms of mental illness comprise bipolar disorder. In other embodiments, the methods further comprise recommending an antidepressant drug to the subject.

Also provided by the present invention are kits determining if a subject is predisposed to major depressive disorder, comprising: at least one reagent capable of specifically detecting at least one polymorphism in an adenylyl cyclase type 7 allele; and instructions for determining whether a subject is predisposed to major depressive disorder. In some preferred embodiments, the at least one polymorphism is a repeat polymorphism. In some embodiments, the at least one reagent comprises a nucleic acid probe that hybridizes under stringent conditions to a nucleic acid sequence selected from the group consisting of the coding strand of the adenylyl cyclase type 7 gene, and the noncoding strand of the adenylyl cyclase type 7 gene. In other embodiments, the at least one reagent comprises a sense primer and an antisense primer flanking the at least one polymorphism in the adenylyl cyclase type 7 allele. In some preferred embodiments, at least one of the primers comprises a fluorescent tag. Additionally, in some preferred embodiments, the instructions comprise instructions required by the United States Food and Drug Administration for use with in vitro diagnostic products. Moreover, the present invention provides kits that further comprise at least one reagent capable of specifically detecting at least one polymorphism in an additional allele associated with major depressive disorder. In some embodiments, the additional allele is in linkage disequilibrium with AC7.R7, while in further embodiments, the additional allele is not in linkage disequilibrium with AC7.R7.

The invention also provides methods of screening compounds, comprising: providing: i) at least one cell comprising an adenylyl cyclase type 7 allele with a tetranucleotide repeat polymorphism, and ii) one or more test compounds; and contacting the at least one cell with the test compound; and detecting a change in adenylyl cyclase type 7 in the at least one cell in the presence of the test compound relative to the absence of the test compound. In some embodiments, the detecting comprises detecting a change in adenylyl cyclase type 7 mRNA. In other embodiments, the detecting comprises detecting a change in adenylyl cyclase type 7 polypeptide. In further embodiments, the detecting comprises detecting a change in adenylyl cyclase type 7 enzymatic activity. In some embodiments, the cell is a platelet. Additionally, in some embodiments, the test compound comprises a drug.

Furthermore, the present invention provides methods of identifying individuals predisposed to major depressive disorder comprising: providing a nucleic acid sample from a subject, the sample containing an adenylyl cyclase type 7 allele; and correlating the identity of the adenylyl cyclase type 7 allele with a predisposition to major depressive disorder. In some preferred embodiments, the subject is Caucasian. In particularly preferred embodiments, the subject is female. In other preferred embodiments, the subject is alcohol-dependent. The present invention provides embodiments wherein the identity of the adenylyl cyclase type 7 allele is accomplished using at least one technique selected from the group consisting of polymerase chain reaction, heteroduplex analysis, single stand conformational polymorphism analysis, ligase chain reaction, comparative genome hybridization, Southern blotting and sequencing. In some embodiments, the nucleic acid sample is selected from the group consisting of buccal cells, biopsy material and blood. In some preferred embodiments, the methods further comprise providing a diagnosis to the subject based on the presence or absence of the polymorphism. In particularly preferred embodiments, the diagnosis differentiates major depressive disorder from other forms of mental illness. In further embodiments, the other forms of mental illness comprise bipolar disorder. In other embodiments, the methods further comprise recommending an antidepressant drug to the subject. Additionally, in some embodiments, the present invention encompasses other polymorphic regions of DNA in the vicinity of the AC7 gene that are in linkage disequilibrium with AC7 (i.e., part of the AC7 haplotype). Thus in some instances, the methods and compositions of the present invention comprise polymorphisms in genes which flank AC7 or are within 10 cM of the 16q12 region as markers of major depressive disorder.

### DESCRIPTION OF THE FIGURES

FIG. 1 schematically illustrates the location of the adenylyl cyclase type 7 (AC7) gene on human chromosome 16. The AC7 gene location is centered at q12.2, as indicated by the black vertical bar designating the confidence interval for the gene location.

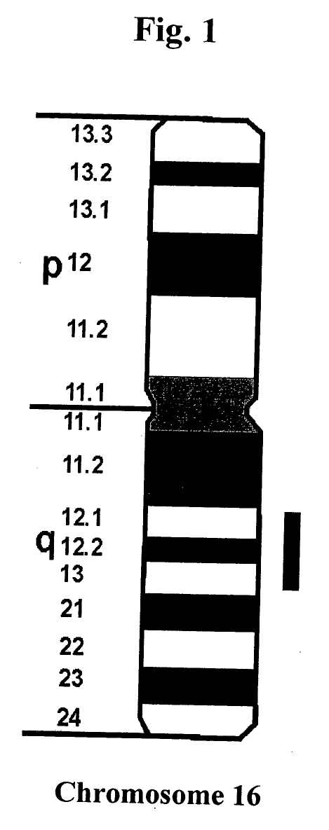

FIG. 2 illustrates the size of the AC7 gene on human chromosome 16. As shown in this figure, the tetranucleotide repeat of interest (i.e., AACA) is located in the 3′ untranslated region of the gene product. Also shown is the allele frequency of the common length polymorphisms in the AC7 tetranucleotide repeat region.

FIG. 3 depicts the results of a PCR analysis for length polymorphisms in the AC7 gene sequence containing the tetranucleotide repeats. Panel A shows the approximate size (i.e., 204 bp) of the PCR fragments obtained from an individual homozygous for the AC7.R7 polymorphism ([AACA]₇; disclosed herein as SEQ ID NO:2). Panels B and C show the size of the PCR fragments obtained from individuals heterozygous for the AC7.R7 polymorphism.

FIG. 4 depicts the forskolin-stimulated platelet AC activity of non-depressed subjects and depressed subjects. The AC activity of subjects lacking an AC7.R7 allele is shown in black, while the AC activity of subjects possessing at least one AC7.R7 allele is shown in white.

### GENERAL DESCRIPTION OF THE INVENTION

Adenylyl cyclase type 7 (AC7) was considered as a candidate gene for an association study between the known polymorphisms in the AC7 gene structure and major depressive disorder. A structured interview was used to obtain extensive information on the subjects, including DSM-IV diagnosis of major depressive disorder, bipolar disorder, anti-social personality disorder, alcoholism, and family history of mental and addictive disorders. Blood was obtained from each subject. Statistical analysis was performed with genotypic data from Caucasian male and female subjects (n=745) from Montreal, Canada; Sydney, Australia; and Helsinki, Finland (See, Table 1).

DNA was genotyped for a tetranucleotide repeat polymorphism in AC7. A logistic regression analysis across all subjects revealed a significant association between major depressive disorder in subjects with a family history of depression (familial depression), and genotypes containing the seven repeat (R7) polymorphism in AC7 (p<0.02). No other significant associations were noted with thirty-two other demographic and mental health variables (See, Table 2). Individuals with the AC7.R7 allele were 2.4 times more likely to have familial depression. When women were analyzed separately from men, the statistical significance of the allelic association (p<0.008), and the odds ratio (O.R.=2.6) was higher for women compared to men (p=0.27; O.R.=1.5). When women were categorized as DSM-IV alcohol-dependent vs. non-alcohol dependent, those with a diagnosis of alcohol dependence were found to have a likelihood (O.R of 4.6 (p<0.002) for familial depression if they had the AC7.R7 allele. Thus, the AC7.R7 allele seems to well identify individuals (primarily, women) who may be predisposed to depression, particularly if they are alcohol-dependent.

Definitions

To facilitate understanding of the invention, a number of terms are defined below.

The terms “subject” as used herein, refers to a human. It is intended that the term encompass healthy individuals, as well as, individuals predisposed to, or suspected of having a major depressive disorder. Typically, the terms “subject” and “patient” are used interchangeably. In some preferred embodiments of the present invention, the term subject refers to specific subgroups of patients including but not limited to Caucasians, females, and alcohol-dependent individuals. As used herein, the term “Caucasian” refers to a member of the white race consisting of individuals of European, north African, or southwest Asian ancestry. The term “female” encompasses both women and girls. As used herein, the term “alcohol-dependent” refers to an individual addicted to alcohol.

As used herein, the terms “adenylyl cyclase” and “adenylate cyclase” refer to a class of enzymes responsible for the catalysis of cAMP from ATP. In preferred embodiments, the terms “adenylyl cyclase 7” and “AC7” refer to human adenylyl cyclase type VII.

The term “gene” refers to a nucleic acid (e.g., DNA) sequence that comprises coding sequences necessary for the production of a polypeptide (e.g., AC7), precursor, or RNA (e.g., mRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid” refers to any nucleic acid containing molecule, including but not limited to, DNA, cDNA and RNA. In particular, the terms “AC7 gene” and “AC7 nucleic acid” refer to the full-length AC7 nucleotide sequence (e.g., contained in human chromosome 16 from bp 37,275,848 to bp 37,348,868). The terms “AC7 gene” and “AC7 nucleic acid” as used herein, also encompass fragments of the AC7 sequence, as well as other domains within the full-length AC7 nucleotide sequence. Furthermore the term “AC7 nucleotide sequence” encompasses DNA, cDNA, and RNA (e.g., GenBank Accession No. NM_{—}001114; and SEQ ID NO:1) sequences.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the term “portion of a chromosome” refers to a discrete section of the chromosome. Chromosomes are divided into sites or sections by cytogeneticists as follows: the short (relative to the centromere) arm of a chromosome is termed the “p” arm; the long arm is termed the “q” arm. Each arm is then divided into two regions termed region 1 and region 2. Region 1 is closest to the centromere. Each region is further divided into bands. The bands may be further divided into sub-bands. For example, the 16q12.2 portion of human chromosome 16 is the portion located on the long arm (q) in the first region (1) in the 2nd band (2) in sub-band 2 (0.2).

The term “wild-type” refers to a gene or gene product isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “polymorphism” refers to the regular and simultaneous occurrence in a single interbreeding population of two or more alleles of a gene, where the frequency of the rarer alleles is greater than can be explained by recurrent mutation alone (typically greater than 1%). In preferred embodiments, the term “polymorphism” refers to a repeat polymorphism in the 3′ untranslated region of AC7. In particularly preferred embodiments, the repeat polymorphism is a tetranucleotide repeat polymorphism, [AACA]₇, which is associated with a predisposition to a major depressive disorder. In other embodiments, the term “polymorphism” refers to a repeat polymorphism in the 3′ untranslated region of AC7, [AACA]₅, which is associated with protection from major depressive disorder. In other embodiments, the term “polymorphism” refers to a functional polymorphism in the promoter region of the serotonin transporter gene (SLC6A4).

The terms “repeat polymorphism” and “microsattelite repeat” as used herein refer to a variety of simple di- (dinucleotide repeats), tri- (trinucleotide repeats), tetra-, and pentanucleotide tandem repeats that are dispersed in the euchromatic arms of most chromosomes.

As used herein, the term “allele” refers to one of at least two mutually exclusive forms of the same gene, occupying the same locus on homologous chromosomes, and governing the same biochemical and developmental process.

The term “additional allele” as used herein, refers to a form of a gene other than AC7.R7 allele, which is associated a subject's predisposition to major depressive disorder. In some embodiments, the additional allele comprises the “short” or “s” allele of the serotonin transporter gene-linked polymorphic region (5-HTTLPR). Two copies of the short 5-HTTLPR allele (s/s) are known in the art to predispose individuals to depressive symptoms, diagnosable depression, and/or suicidality (See, Caspi et al., Science, 301:386-389, 2003; and Holden, Science, 301:292-293, 2003).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization, 1985). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1X SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5× SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “sense primer” refers to an oligonucleotide capable of hybridizing to the noncoding strand of gene. The term “antisense primer” refers to an oligonucleotide capable of hybridizing to the coding strand of a gene.

As used herein, the term “fluorescent tag” refers to a molecule having the ability to emit light of a certain wavelength when activated by light of another wavelength. “Fluorescent tags” suitable for use with the present invention include but are not limited to fluorescein, rhodamine, Texas red, 6-FAM, TET, HEX, Cy5, Cy3, and Oregon Green.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” as used herein, refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “ligase chain reaction” and “ligase amplification reaction” refer to methods for detecting small quantities of a target DNA, with utility similar to PCR. Ligase chain reaction relies on DNA ligase to join adjacent synthetic oligonucleotides after they have bound the target DNA. Their small size means that they are destabilized by single base mismatches and so form a sensitive test for the presence of mutations in the target sequence.

The terms “single-strand conformation polymorphism” and “SSCP,” as used herein, refer to the ability of single strands of nucleic acid to take on characteristic conformations under non-denaturing conditions, which in turn can influence the electrophoretic mobility of the single-stranded nucleic acids. Changes in the sequence of a given fragment (i.e., mutations) will also change the conformation, consequently altering the mobility and allowing this to be used as an assay for sequence variations (Orita et al., Genomics 5:874-879, 1989).

As used herein, the terms “conformation-sensitive gel electrophoresis” or “CSGE” refer to methods for detecting mutations involving distinguishing DNA heteroduplexes from homoduplexes via mildly denaturing gel electrophoresis. CSGE protocols are well known in the art (Ganguly et al., Proc Natl Acad Sci USA 90:10325-10329, 1993).

The term “DNA sequencing” refers to methods used to determine the order of nucleotide bases in a DNA molecule or fragment. The term “DNA sequencing” includes for example, dideoxy sequencing and Maxam-Gilbert sequencing.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness (e.g., major depressive disorder), sickness, or disorder of bodily function. Test compounds comprise both known and potential therapeutic compounds. A test compound can be determined to be therapeutic by screening using the screening methods of the present invention.

The term “change” as used herein refers to a difference or a result of a modification or alteration. In preferred embodiments, the term “change” refers to a measurable difference between states (e.g., higher or lower AC7 mRNA or protein expression in a cell in the presence and absence of a test compound). In some embodiments, the change is at least 10%, preferably at least 25%, more preferably at least 50%, and most preferably at least 90% more or less than that of a control condition.

As used herein, the term “sample” is meant to include a specimen obtained from subject. The term “sample” encompasses fluids, solids, and tissues. In preferred embodiments, the term “sample” refers to blood or biopsy material obtained from a living body for the purpose of examination via any appropriate technique (e.g., needle, sponge, scalpel, swab, etc.). In particularly preferred embodiments, the term “sample” refers to buccal cells (e.g., cells of the inner lining of the mouth or cheek). Buccal cell samples are obtained using any suitable method, including but not limited to collection via tongue depressor, cytobrush or mouthwash (See, Moore et al., Biomarkers, 6:448-454, 2001).

The term “bipolar disorder” as used herein, refers to a form of mood disorder characterized by a variation of mood between a phase of manic or hypomanic elation, hyperactivity and hyper imagination, and a depressive phase of inhibition, slowness to conceive ideas and move, and anxiety or sadness. Together these form what is commonly known as manic depression.

The term “depression” as used herein, refers to a mental state of depressed mood characterized by feelings of sadness, despair and discouragement. Depression ranges from normal feelings of the blues through dysthymia to major depression.

As used herein, the terms “major depression” and “major depressive disorder” refers to a clinical syndrome (See, DSM-IV) that includes a persistent sad mood or loss of interest in activities that persists for at least 2 weeks in the absence of external precipitants. “Major depression” is distinct from a grief reaction brought on for instance by the death of a loved one. Symptoms of depression may include any of the following: problems concentrating, remembering, and/or making decisions, changes in eating and/or sleeping habits, a loss of interest in enjoyable activities, difficulty going to work or taking care of daily responsibilities, feelings of guilt and/or hopelessness, slowed thoughts and/or speech, and preoccupation with thoughts of death or suicide.

As used herein, the term “risk of developing major depressive disorder” refers to a subject's relative risk (e.g., the percent chance or a relative score) of developing depression during their lifetime.

The term “subject suspected of having depression” refers to a subject that presents one or more symptoms indicative of a depression (e.g., unexplained insomnia, fatigue, irritability, etc.) or is being screened for depression (e.g., during a routine physical).

As used herein, the term “diagnosis” refers to the determination of the nature of a case of disease. In some preferred embodiments of the present invention, methods for making a diagnosis are provided which permit major depressive disorder to be distinguished from other forms of mental illness including but not limited to manic depression (bipolar disorder), schizophrenia, attention deficit disorder, and obsessive compulsive personality.

The term “reagent(s) capable of specifically detecting a tetranucleotide repeat polymorphism in an AC7 allele” refers to reagents used to detect the polymorphism in question from an AC7 gene, cDNA, or RNA. Examples of suitable reagents include but are not limited to, nucleic acid probes capable of specifically hybridizing to AC7 mRNA or cDNA.

As used herein, the term “instructions for determining whether a subject is predisposed to major depressive disorder” refers to instructions for using the reagents contained in the kit for the detection and characterization of an AC7 allele in a sample from a subject. In some embodiments, the instructions further comprise the statement of intended use required by the U.S. Food and Drug Administration (FDA) in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

### DESCRIPTION OF THE INVENTION

The present invention relates to compositions and methods for determining whether an individual is predisposed to major depression. In particular, the present invention relates to a repeat polymorphism in the adenylyl cyclase type VII (AC7) gene. The present invention encompasses methods of identifying alleles of the AC7 gene bearing the repeat polymorphism (designated herein as the R7 allele), as well as the expression of the AC7.R7 allele in transgenic organisms and in prokaryotic and eukaryotic cell culture systems. Additionally, methods for identifying drugs that inhibit or potentiate the activity of the R7 allele of the AC7 gene or mRNA are encompassed by the present invention. Thus, the present invention provides a genetic marker useful for the diagnosis, characterization and treatment of major depression.

The search for the genetic determinants of affective illness and, particularly, major depression, has produced proposals for a number of markers and candidate genes, which still require verification in independent studies (Stoltenberg and Burmeister, Human Molecular Genetics, 9:927-935, 2000). Two genetic study approaches have been applied to the investigation of the determinants of major depressive disorder and other affective disorders. Linkage studies, which utilize a family design, and association studies, which are usually performed with groups of unrelated individuals, have been typically used for investigations of affective illness phenotypes. In general, linkage studies are powerful for detecting genetic loci in single gene disorders. On the other hand, properly structured association studies have more statistical power to identify genetic loci which may, in and of themselves, have a relatively modest contribution, but in aggregate are important as determinants of polygenic disorders (Tabor et al., Nat Rev Genet 3:391-397, 2002; and Cardon and Bell, Nat Rev Genet, 2:91-99, 2001).

The basis for choosing a candidate gene for an association study is usually that the gene of interest has an evident functional link to the disease, and evidence exists that the polymorphisms in the gene of interest may be in linkage disequilibrium (i.e., linked) with the disease phenotype. Although monoaminergic transmitter synthesis and degradation enzymes, and monoamine receptors and transporters have played prominent roles in the choice of candidate genes for association studies with major depressive disorder (Johansson et al., Eur Neuropsychopharmacol, 11:385-394, 2001), current research is focusing attention on second-messenger signaling systems (e.g., c-AMP) and transcription factors (e.g., CREB) as likely candidates for association studies in the area of major depressive disorders (Nestler et al., Neuron, 34:13-25, 2002).

Cyclic AMP (c-AMP) is an intracellular messenger that is produced by the actions of the enzyme, adenylyl cyclase. Adenylyl cyclase (AC) activity is controlled by numerous factors, including the guanine nucleotide-binding proteins (G proteins). AC activity can also be directly stimulated by the binding of the plant alkaloid, forskolin, to the AC enzyme. In a recent examination of subjects with a history of major depression and matched controls, we found that forskolin-stimulated AC activity in platelets of the depressed subjects was significantly lower than forskolin-stimulated AC activity in control subjects (Menninger and Tabakoff, Biol Psychiatry, 42:30-38, 1997). An earlier study (Cowbum et al., Brain Res, 633:297-304, 1994), also demonstrated that both forskolin and guanine nucleotide (GppNHp)-stimulated AC activity was lower in membranes prepared postmortem from the brains of depressed subjects who had committed suicide. A relationship between AC activity and major depression is not only evident from studies of AC activity in platelets or post-mortem brain of depressive human subjects, but is also evident from studies of administration of antidepressants or electroconvulsive shock (ECS) to animals (Ozawa and Rasenick, Mol Pharmacol 36:803-808, 1989; and Ozawa and Rasenick, J Neurochem, 56:330-338, 1991). An increase in Gs protein-stimulated AC activity in brain resulted from the chronic treatment of rats with several of the antidepressant medications or ECS.

We have previously demonstrated that the major isoform of AC in platelet precursor cells (megakaryocytes) is the Type 7 AC (AC7, Hellevuo et al., Biochem Biophys Res Commun 192:311-318, 1993). AC7 is also found in brain, predominantly distributed in Golgi Type 1 and Golgi Type 2 GABA neurons (Mons et al., Brain Res, 788-251-262, 1998). We have also described a tetranucleotide repeat polymorphism in the 3′ untranslated region of the cDNA for AC7 (Hellevuo et al., Amer J Med Gen, 74:95-98, 1997), and have localized the gene for AC7 to human chromosome 16 (16q12). This region has been shown to contain genes (16q23) for human haptoglobin protein variants that appear to be associated with major depressive illness (Maes et al., Am J Psychiatry, 42:30-38, 1994). However, prior to the studies conducted during development of the present invention, polymorphisms in the AC7 gene, which are associated with major depressive illness, had not been identified.

I. Identification of an Association Between AC7.R7 and Major Depressive Disorder

During the development of the present invention, AC7 was selected as a candidate gene for an association study between known polymorphisms in AC7 and major depressive disorder. Table 1 shows study population characteristics with regards to race, gender, location of the individuals used for the study, and the psychiatric diagnoses present in the study population. Only the white (Caucasian) individuals were used for association analysis.

Table 3 presents the results of a logistic regression analysis, which initially utilized the thirty-two variables listed in Table 2, to explore the odds of having an AC7.R7 allele and a given phenotype. A number of the variables were removed prior to the model building process. The variables listed as phenotypes in Table 3 represent the components in the final model of main effects. In examining the P values, a statistically significant association is evident between the AC7.R7 allele and the platelet forskolin-stimulated adenylyl cyclase activity determined for each subject. The phenotype abbreviations used in Table 3, are defined in the legend at the bottom of the table.

Additionally, there is a significant association between familial depression (DEPXFAM) and the AC7.R7 genotype. We had previously demonstrated that forskolin-stimulated adenylyl cyclase activity in platelets is related to depression (Menninger and Tabakoff, Biol Psychiatry 42:30-38, 1997). Now, during the course of development of the present invention, we have demonstrated that familial depression, as well as, the platelet forskolin-stimulated adenylyl cyclase activity, are associated with the AC7.R7 genotype (Table 3).

TABLE 1
Study Population Characteristics

| | | Adjusted |
|---|---|---|
| Characteristic | Number | Number^{#} |
| White | 745 | |
| Black | 14 | |
| Asian/Indian | 41 | |
| Male | 660 | 540 |
| Female | 225 | 206 |
| Montreal | 449 | 406 |
| Helsinki | 131 | 130 |
| Sydney | 245 | 210 |
| Alcohol-Dependent | 438 | |
| Non-Alcohol-Dependent | 377 | |
| Depressed | 166 | 157 |
| Non-Depressed | 659 | 589 |
| Antisocial Personality (ASP) | 161 | 150 |
| Non-ASP | 664 | 596 |
| Familial Depression* | 69 | 68 |
| No familial Depression | 573 | |

*Depression in proband and first-degree relative.
^{#}Individuals who did not provide reliable information on depression in first-degree relatives were excluded from this group.

TABLE 2
Variables

| | |
|---|---|
| GENDER | Sex |
| AGE | Age (years) |
| EXERREGG | Regular Exercise |
| SMOKTYPE | Smoking recode |
| DRINKERS | Drinking recode |
| CONQUART | Alcohol consumption in quartiles (gm/day) |
| AD_LIFE | Lifetime alcohol dependence |
| AB_LIFE | Lifetime alcohol abuse |
| FHX1AD | Family history of alcohol dependence in 1st degree |
| | relative, without clustering |
| FHX1ADC | Family history of alcohol dependence in 1st degree |
| | relative, with clustering |
| DEPRES4F | DSM-IV major depression during subject's lifetime |
| FHX_DEP1 | Family history of depression in 1st degree relative |
| DEPFXFAM | Family history of depression in 1st degree relative and |
| | diagnosis of depression in subject at any point in |
| | lifetime |
| ANTIDP30 | Used any antidepressant in last month |
| OTHER30 | Used medication other than antidepressants in last month |
| ANXIETY | Ever seek treatment for anxiety |
| CONDUCT | Conduct disorder (DSM-IV) |
| ANTISOC | Antisocial personality disorder (DSM-IV) |
| MJA_LIFE | Lifetime marijuana abuse |
| AC.VII.G | AC-VII genotypes |
| AC.VIIA1 | AC-VII allele (AACA) 5 |
| AC.VIIA2 | AC-VII allele (AACA) 6 |
| AC.VIIA3 | AC-VII allele (AACA) 7 (AC7.R7) |
| FOR_AC | Forskolin-stimulated AC activity |
| FOR_AC_4 | Forskolin-stimulated AC activity (quartiles) |
| AC.IX.G | AC-IX Genotypes |
| AC.IX.A1 | AC-IX allele (TAA) 8 |
| AC.IX.A2 | AC-IX allele (TAA) 9 |
| AC.IX.A3 | AC-IX allele (TAA) 10 |
| AC.IX.A4 | AC-IX allele (TAA) 11 |
| AC.IX.A5 | AC-IX allele (TAA) 12 |
| AC.IX.A6 | AC-IX allele (TAA) 13 |

Given the epidemiological data indicating that females are more prone to major depressive disorder as compared to males, a logistic regression analysis was carried out separately on male and female subjects. Table 4 demonstrates that when males and females are analyzed separately, the statistical significance of the association between forskolin-stimulated adenylyl cyclase activity and the AC7.R7 allele in males becomes marginal, and that in males the statistically significant association between familial depression and AC7.R7 allele is lost. On the other hand, in males, a statistically significant association is realized between the AC7.R7 allele and the variable AD_life, which denotes a DSM-IV diagnosis of alcohol dependence some time during the individual's lifetime. In females, however, the statistically significant association between familial depression and the AC7.R7 allele remained significant when analyzed separately from the males, as shown in Table 5.

Table 6 presents the results of analysis using Pearson's χ² and the calculations of odds ratios which describe the “chance” that an individual with familial depression will also have the AC7.R7 allele genotype. Odds ratios can be interpreted as the “chance” that an individual with a particular phenotype will also have the AC7.R7 genotype or as the chance that the AC7.R7 allele-carrying individuals will display a particular phenotype. The higher the odds ratio, the higher are the “chances” that you can predict phenotype or genotype when you already know one of these variables and are trying to predict the other. Table 6 demonstrates that if one takes the general population, and genotypes the individuals, and bases the prediction of an individual having familial depression on the fact that an individual has an AC7.R7 genotype, one would be 1.9-2.4 fold more likely to predict the phenotype correctly by knowing the genotype, than one would be likely to predict the phenotype by chance alone.

TABLE 3
Logistic Analysis with AC7.R7 Outcome

| Phenotype | Coef. | Std. Err. | z | P > \|z\| |
|---|---|---|---|---|
| Ifor_a_2 | .495 | .235 | 2.09 | 0.036 |
| Ifor_a_3 | .298 | .235 | 1.27 | 0.204 |
| Ifor_a_4 | .489 | .239 | 2.04 | 0.041 |
| depres4F | −.090 | .255 | −0.35 | 0.723 |
| Fhx_dep1 | −.141 | .271 | −0.52 | 0.603 |
| depxfam | 1.090 | .454 | 2.40 | 0.016 |
| ad_life | −.312 | .164 | −0.89 | 0.058 |
| _cons | .112 | .518 | 0.21 | 0.828 |

Legend
Ifor_a_2 lower quartile for forskolin-stimulated AC activity
Ifor_a_3 median quartile for forskolin-stimulated AC activity
Ifor_a_4 highest quartile for forskolin-stimulated AC activity
depres4F DSM-IV major depression
fhx_dep1 Family history of depression in 1st degree relative
depxfam Interaction between family history of depression in 1st degree relative and DSM-IV depression in subject during the subject's lifetime
ad_life Alcohol dependence (DSM-IV) during lifetime
_cons Alcohol consumption in the last 30 days

TABLE 4
Logistic Analysis with AC7.R7 as the Outcome in Males

| Phenotype* | Coef. | Std. Err. | z | P > \|z\| |
|---|---|---|---|---|
| Ifor_a_2 | .541 | .281 | 1.92 | 0.055 |
| Ifor_a_3 | .124 | .274 | 0.45 | 0.649 |
| Ifor_a_4 | .426 | .267 | 1.59 | 0.112 |
| depres4F | .233 | .322 | 0.72 | 0.469 |
| Fhx_dep1 | −.382 | .340 | −1.12 | 0.262 |
| depxfam | .547 | .597 | 0.91 | 0.360 |
| ad_life | −.385 | .191 | −2.01 | 0.044 |
| _cons | .116 | .355 | 0.32 | 0.743 |

*See Legend in Table 3 for phenotype definitions.

TABLE 5
Logistic Analysis with AC7.R7 as the Outcome in Females

| Phenotype* | Coef. | Std. Err. | z | P > \|z\| |
|---|---|---|---|---|
| Ifor_a_2 | .691 | .469 | 1.47 | 0.141 |
| Ifor_a_3 | .839 | .478 | 1.75 | 0.079 |
| Ifor_a_4 | .611 | .607 | −1.00 | 0.315 |
| depres4F | −.392 | .470 | −0.83 | 0.403 |
| Fhx_dep1 | .485 | .499 | 0.97 | 0.330 |
| depxfam | 1.467 | .765 | −1.91 | 0.050 |
| ad_life | −.163 | .358 | −0.45 | 0.649 |
| _cons | −.888 | .667 | −1.33 | 0.183 |

*See Legend in Table 3 for phenotype definitions.

Importantly, the predictive capacity for familial depression of the AC7.R7 genotype is not significant if one is dealing only with males (See, Table 6). However, when one is dealing with only females, the predictive value of the genotype is both highly significant and quite predictive. The odds ratios for females expectedly vary depending on the comparison group being used to juxtapose against the phenotype of familial depression. When familial depression is juxtaposed against any other phenotypes (e.g., normal, abnormal behavior, and/or non-familial depression), the odds ratio is 2.6. When individuals demonstrating familial depression are compared to individuals who show no depression and no family history for depression, the odds ratio increases to 3.0. An even higher odds ratio of 3.3 is generated when one uses the AC7.R7 genotype to distinguish individuals with familial depression from individuals with depression but having no family history of depression (non-familial depression). The odds ratio of 3.3 indicates an excellent predictive capacity for the AC7.R7 genotype when one is trying to ascertain a genetic form of depression (i.e., familial depression) in a group of females who are all suffering from what is diagnosed as a DSM-IV major depressive disorder, but on whom no information is available as to the familial nature of their depressive episodes.

A particular area in need of a reliable, biological marker for the genetic propensity for depression (e.g., familial depression) is in the assessment of alcohol-dependent subjects. A significant number of alcohol-dependent females will display signs of major depressive disorder during the early stages of drinking cessation. A subset of these subjects who demonstrate depressive symptoms are individuals predisposed to familial depression, and these individuals may well respond to antidepressive medication therapy for both their depression and for treatment of their alcohol dependence.

When alcohol-dependent females were assessed for the association of the AC7.R7 allele with familial depression, a highly significant odds ratio of 4.6 was calculated. Thus, one can use the AC7.R7 allele to identify individuals predisposed to familial depression among a group of alcohol-dependent females. Thus, the odds of correctly identifying familial depression on the basis of the AC7.R7 genotype are 4.6 times better than when one has no genotypic information in alcohol-dependent females.

TABLE 6
Odds ratios^{H} for Association of AC7.R7 Allele
with Familial Depression

| | | | | |
|---|---|---|---|---|
| | Pearson's | | lower | upper |
| Group | χ² P value | O.R. | limit | limit |

Familial depression vs all other phenotypes

| | | | | |
|---|---|---|---|---|
| Males and Females | 0.010 | 1.9 | 1.4 | 3.1 |
| (n = 746) | | (2.4)* | (1.4)* | (7.0)* |
| Males (all) | 0.27 | 1.5 | 0.7 | 3.2 |
| (n = 540) | | | | |
| Females (all) | 0.008 | 2.6 | 1.3 | 5.3 |
| (n = 206) | | | | |
| Alcohol-dependent | 0.01 | 2.7 | 1.2 | 6.2 |
| females | | | | |
| (n = 122) | | | | |

Familial depression vs No Depression and No Family History

| | | | | |
|---|---|---|---|---|
| Females (all) | 0.005 | 3.0 | 1.4 | 6.4 |
| (n = 133) | | | | |
| Alcohol-dependent | 0.002 | 4.6 | 1.8 | 12.3 |
| females | | | | |
| (n = 77) | | | | |

Familial depression vs Non-Familial Depression

| | | | | |
|---|---|---|---|---|
| Females (all) | 0.01 | 3.3 | 1.4 | 8.4 |
| (n = 79) | | | | |
| Alcohol-dependent | 0.06 | 2.6 | 0.9 | 7.5 |
| females | | | | |
| (n = 60) | | | | |

^{H}Derived from χ² analysis
*Derived from logistic analysis

Table 7 provides data using another form of statistical analysis for determining the utility of the AC7.R7 genotype as a diagnostic tool for familial depression. The AC7.R7 marker again provides statistically significant specificity and sensitivity for identifying familial depression in females, whether one is attempting to differentiate familially depressed females from all other females in the population, or whether one is trying to distinguish familially depressed females from those females showing no prior history of depression and not having a family history of depression.

In the general population of females, one can also use the AC7.R7 genotype to distinguish familial depression from non-familial depression (specificity is 68%; sensitivity is 62%). Additionally, using ROC analysis to assess the utility of the AC7.R7 allele as a diagnostic tool for familial depression in the alcohol-dependent female population, the specificity of the AC7.R7 allele as a marker for familial depression increases to 75% with no loss in sensitivity.

An odds ratio was also calculated for the relationship in male subjects for the association between the AC7.R7 allele and alcohol dependence (See, Table 6). This odds ratio was 0.7 indicating some protective effect of having the AC7.R7 allele as part of a male's genotype.

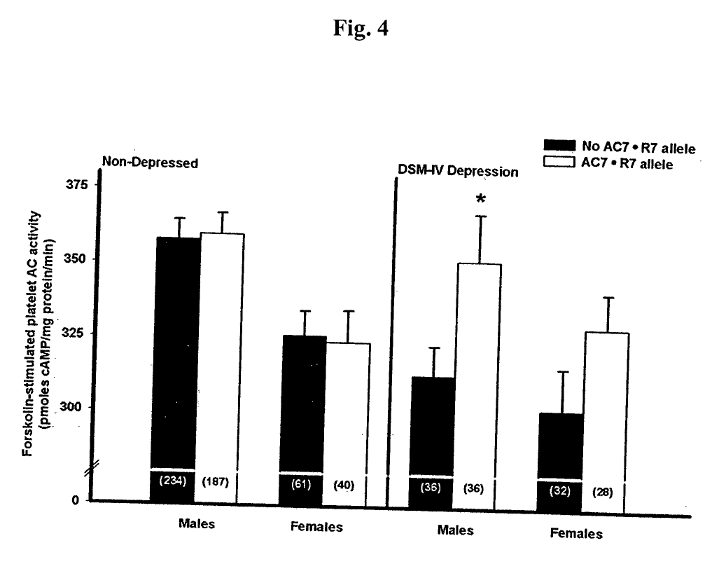

Previously we have demonstrated that depressed subjects have lower forskolin-stimulated, platelet adenylyl cyclase activity (Menninger and Tabakoff, Amer J Med Gen, 74:95-98, 1997), and that platelets contain a preponderance of AC7 (Hellevuo et al., Biochem Biophys Res Commun, 192:311-318, 1993). During development of the present invention, the relationship between lower platelet forskolin activated AC activity and AC7 genotype was explored. As shown in FIG. 4, depressed subjects with the AC7.R7 allele had platelet forskolin-stimulated AC activity that was higher than that of subjects without the AC7.R7 allele (both men and women). Therefore, platelet forskolin-stimulated AC activity measured in combination with genotype information is contemplated to provide additional tools for substantiating DSM-UV-categorized depression.

TABLE 7
Receiver Operating Characteristics (ROC) Analysis for Sensitivity and Specificity
of AC7.R7 as a Diagnostic Tool for Familial Depression

| | | Area | Asymptotic | | |
|---|---|---|---|---|---|
| | Diagnostic | Under | Significance^{a} | | |
| Group | Differentiation | Curve | 2 sided | Specificity | Sensitivity |
| Females | Familial Depression vs | 0.62 | 0.024 | 62 | 62 |
| (n = 206) | All Others | | | | |
| Females | Familial Depression vs | 0.63 | 0.017 | 65 | 62 |
| (n = 133) | No Depression or | | | | |
| | Family History of | | | | |
| | Depression | | | | |
| Females | Familial Depression vs | 0.65 | 0.026 | 68 | 62 |
| (n = 79) | Non-Familial | | | | |
| | Depression | | | | |
| Alcohol-Dependent | Familial Depression vs | 0.62 | 0.120 | 63 | 61 |
| Females | Non-Familial | | | | |
| (n = 60) | Depression | | | | |
| Alcohol-Dependent | Familial Depression vs | 0.68 | 0.008 | 75 | 61 |
| Females | No Depression or | | | | |
| (n = 77) | Family History of | | | | |
| | Depression | | | | |

^{a}Null hypotheses: true area = 0.5

A polychotomous logistic regression analysis was done, to ascertain the effect of treatment with antidepressant agents on platelet forskolin-stimulated AC activity. In particular, if the activity of the various polymorphic forms of AC7 is contributing to the etiology of major depressive illness, than the treatment of the illness with antidepressants may also rectify the lower AC activity exhibited by depressed subjects. The logistic analysis produced the following results. 1) Individuals with a diagnosis of major depressive disorder who did not use antidepressants had significantly lower forskolin-stimulated platelet AC activity than did individuals who were not depressed nor used antidepressants. 2) Individuals with a diagnosis of major depressive disorder who used antidepressants had significantly higher forskolin-stimulated platelet AC activity compared to the depressed subjects (above) who did not use antidepressants. Additionally, when an odds ratio was calculated comparing depressed subjects who were using antidepressants to depressed subjects who were not using antidepressants, it was found that depressed subjects using antidepressants had greater odds of having normal or elevated forskolin-stimulated platelet AC activity. These results demonstrate that the use of antidepressants by depressed individuals can normalize the low forskolin-stimulated AC activity associated with depression. Given the association of the AC7.R7 allele with both familial depression and forskolin-stimulated AC activity, the presence of the AC7.R7 allele can predict predisposition to familial depression and to low forskolin-stimulated AC activity. Additionally, AC7.R7 can be used as a marker for individuals who will respond to antidepressants with an increase in AC activity, as well as a decrease in depressive signs and symptoms.

II. Detection of AC7 Alleles

A. AC7 Alleles

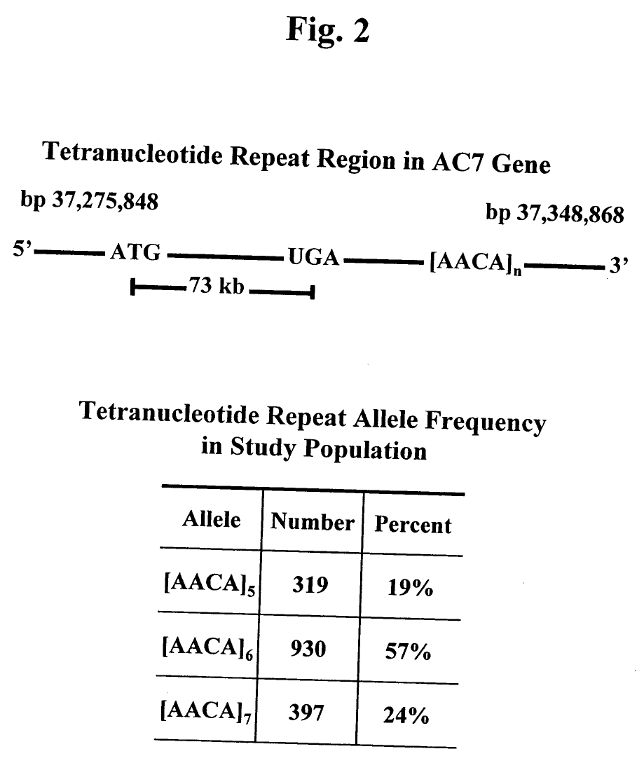

In some embodiments, the present invention includes alleles of AC7 that increase or decrease a subject's susceptibility to major depressive disorder (e.g., including, but not limited to, AC7.R7 and AC7.R5). Analysis of naturally occurring human AC7 alleles revealed that patients with increased susceptibility to major depressive disorder have an AC7 allele that contains an a seven tetranucleotide repeat in the 3′ untranslated region (e.g., [AACA]₇ disclosed herein as SEQ ID NO:2). However, the present invention is not limited to this seven tetranucleotide repeat polymorphism. In fact, any AC7 polymorphism and any polymorphism in linkage with the AC7 polymorphism that are associated with predisposition to or protection from major depressive disorder, are within the scope of the present invention. For example, in some embodiments, the present invention provides single-nucleotide polymorphisms of AC7, while in other embodiments, five or six tetranucleotide repeat polymorphisms are provided (See, FIG. 2).

B. Detection of AC7 Alleles

Accordingly, the present invention provides methods for determining whether a patient has an increased susceptibility to major depressive disorder by determining whether the individual has an AC7.R7 allele. In other embodiments, the present invention provides methods for providing a prognosis of increased risk for major depressive disorder to an individual based on the presence or absence of one or more polymorphisms in the AC7 gene. In preferred embodiments, the polymorphism causes or contributes to major depressive disorder.

A number of methods are available for analysis of polymorphisms. Assays for detection of polymorphisms or mutations fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

1. Direct sequencing Assays

In some embodiments of the present invention, polymorphisms are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the polymorphism of interest) is sequenced using any suitable method, including but not limited to manual sequencing using radioactive marker nucleotides, or automated sequencing. The results of the sequencing are displayed using any suitable method. The sequence is examined and the presence or absence of a given polymorphism is determined.

2. PCR Assay

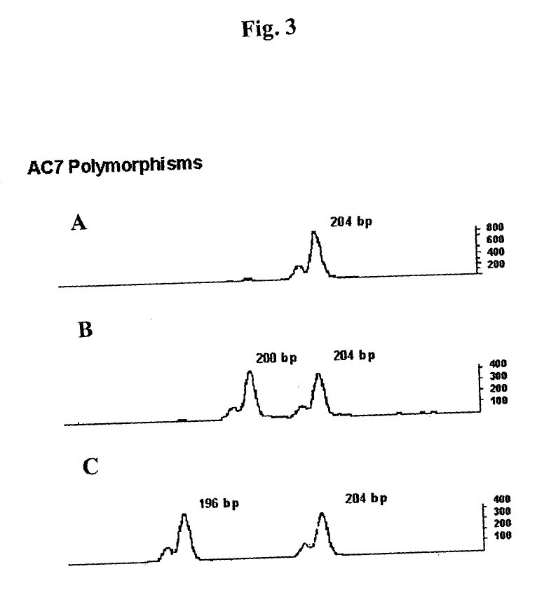

In some embodiments of the present invention, polymorphisms are detected using a PCR-based assay. In some embodiments, the PCR assay comprises the use of oligonucleotide primers to amplify an AC7 fragment containing the repeat polymorphism of interest. The presence of an additional repeat in the AC7 gene results in the generation of a longer PCR fragment which can be detected by gel electrophoresis. For instance, by use of the method described in Example 5, the AC7.R7 allele is detected by the appearance of a 204 bp PCR product, while the AC7.R6 and AC7.R5 alleles are detected by the appearance of a shorter 200 and 196 bp PCR products, respectively (See, FIG. 3).

In other embodiments, the PCR assay comprises the use of oligonucleotide primers that hybridize only to the mutant or wild type allele of AC7 (e.g., to the region of polymorphism). Both sets of primers are used to amplify a sample of DNA. If only the mutant primers result in a PCR product, then the patient has the mutant AC7 allele. If only the wild-type primers result in a PCR product, then the patient has the wild type allele of AC7.

3. Fragment Length Polymorphism Assays

In some embodiments of the present invention, polymorphisms are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

a. RFLP Assay

In some embodiments of the present invention, polymorphisms are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

b. CFLP Assay

In other embodiments, polymorphisms are detected using a CLEAVASE fragment length polymorphism assay (CFLP; Third Wave Technologies, Madison, Wis.; See e.g., U.S. Pat. No. 5,888,780). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

4. Hybridization Assays

In preferred embodiments of the present invention, polymorphisms are detected by hybridization assay. In a hybridization assay, the presence of absence of a given polymorphism or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., polymorphism) is detected directly by visualizing a bound probe (e.g., a Northern or Southern assay; See e.g., Ausabel et al (eds.), Current Protocols in Molecular Biology, John Wiley & Sons, NY, 1991). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

b. Detection of Hybridization Using “DNA Chip” Assays

In some embodiments of the present invention, polymorphisms are detected using a DNA chip hybridization assay. In this assay, a series of oligonucleotide probes are affixed to a solid support. The oligonucleotide probes are designed to be unique to a given polymorphism. The DNA sample of interest is contacted with the DNA “chip” and hybridization is detected.

In some embodiments, the DNA chip assay is a GeneChip (Affymetrix, Santa Clara, Calif.; See e.g., U.S. Pat. No. 6,045,996) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

In other embodiments, the DNA chip assay is CodeLink-type Bioarray (Amersham Biosciences, Piscataway, N.J.) assay. The CodeLink Bioarray technology employs oligonucleotide probes that are piezo-electrically deposited onto a proprietary 3-D aqueous gel matrix, to mimic solution-phase hybridization kinetics.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. No. 6,068,818). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding, In still further embodiments, an array technology based upon the segregation of fluids on a flat surface (chip) by differences in surface tension (ProtoGene, Palo Alto, Calif.) is utilized (See e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removed by spinning.

DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

C. Enzymatic Detection of Hybridization

In some embodiments of the present invention, genomic profiles are generated using a assay that detects hybridization by enzymatic cleavage of specific structures (INVADER assay, Third Wave Technologies; See e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified genomic DNA. The isolated DNA sample is contacted with the first probe specific either for a SNP/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. No. 5,952,174). In this assay, SNPs are identified using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

5. Mass Spectroscopy Assay

In some embodiments, a MassARRAY system (Sequenom, San Diego, Calif.) is used to detect polymorphisms (See e.g., U.S. Pat. No. 6,043,031). DNA is isolated from blood samples using standard procedures. Next, specific DNA regions containing the polymorphism of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization—Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

6. Kits for Analyzing Risk of Major Depressive Disorder

The present invention also provides kits for determining whether an individual contains a specific AC7 polymorphism. In some embodiments, the kits are useful in determining whether the subject is at risk of developing major depressive disorder. The diagnostic kits are produced in a variety of ways. In some embodiments, the kits contain at least one reagent for specifically detecting a mutant AC7 allele. In preferred embodiments, the kits contain reagents for detecting an AACA repeat polymorphism in the AC7 gene. In preferred embodiments, the reagents are primers for amplifying the region of DNA containing the repeat polymorphism. In other preferred embodiments, the reagent is a probe that binds to the polymorphic region. In some embodiments, the kit contains instructions for determining whether the subject is at risk for developing major depressive disorder. In preferred embodiments, the instructions specify that risk for developing major depressive disorder is determined by detecting the presence or absence of a mutant AC7 allele in the subject, wherein subjects having an allele containing a [AACA]₇ repeat have an increased risk of developing major depressive disorder. In some embodiments, the kits include ancillary reagents such as buffering agents, nucleic acid stabilizing reagents, protein stabilizing reagents, and signal producing systems (e.g., fluorescence generating systems). The test kit may be packaged in any suitable manner, typically with the elements in a single container or various containers as necessary along with a sheet of instructions for carrying out the test. In some embodiments, the kits also preferably include a positive control sample.

In further preferred embodiments, the at least one reagent for detecting an AACA repeat polymorphism within the AC7 gene is combined with at least one reagent suitable for detecting at least one polymorphism in an additional allele associated with major depressive disorder. In some embodiments, the additional allele comprises the short allele of the serotonin transporter (See, Caspi et al., Science, 301:386-389, 2003; and Holden, Science, 301:292-293, 2003).

7. Bioinformatics

In some embodiments, the present invention provides methods of determining an individual's risk of developing major depressive disorder based on the presence of one or more mutant alleles of AC7. In other embodiments, the information on the presence or absence of one or more alleles of AC7 is combined with data on the presence or absence of other polymorphisms for determining an individual's risk of developing a major depressive disorder. In some embodiments, the analysis of polymorphism data is automated. For example, in some embodiments, the present invention provides a bioinformatics research system comprising a plurality of computers running a mullet-platform object oriented programming language (See e.g., U.S. Pat. No. 6,125,383). In some embodiments, one of the computers stores genetics data (e.g., the risk of contacting major depressive disorder associated with a given polymorphism). In some embodiments, one of the computers stores application programs (e.g., for analyzing transmission disequilibrium data or determining genotype relative risks and population attributable risks). Results are then delivered to the user (e.g., via one of the computers or via the internet).

III. Other Utilities

The utility of genotyping individuals for the AC7.R7 allele when one wishes to identify individuals (particularly, females) who may be predisposed to a familial form of depression is evident from each of the statistical evaluations of association performed during development of the present invention. An even greater utility may be derived when one is interested in assessing the predisposition to familial depression in subpopulations of females. This is particularly true when using the AC7.R7 allele as an aid in diagnosis of familial depression within an alcohol-dependent group of females.

The utility of utilizing a genetic marker such as AC7.R7 as a component of a diagnostic approach for major depressive disorder is that the genetic marker may also assist in making appropriate treatment decisions. It is contemplated, that screening for the presence of the AC7.R7 allele will aid physicians in distinguishing major depressive disorder of a familial nature, from bipolar disorder or generalized anxiety disorders, which do not have a statistically significant association with the AC7.R7 allele. Importantly, medications used for treating major depressive disorder versus bipolar (manic-depressive) disorder and generalized anxiety syndromes are quite different, even though all three of these disorders present with overlapping symptomology. Moreover, major depressive disorder appears to be a heterogeneous entity, since different subgroups diagnosed with this disorder respond differentially to particular medications. Thus, it is contemplated that screening for the AC7.R7 allele will prove useful in defining subtypes of major depressive disorder patients who can be successfully treated with particular classes of medications. Additionally, genotyping individuals who participate in clinical trials of novel antidepressants, is contemplated to provide relevant information for assessing drug efficacy.

Experimental

The following examples are provided in order to demonstrate and further illustrate certain preferred embodiments and aspects of the present invention and are not to be construed as limiting the scope thereof.

In the experimental disclosure which follows, the following abbreviations apply: AC (adenylyl cyclase); AC7 (AC type VII); AC7.R7 ([AACA]₇ repeat polymorphism in AC7 3′ untranslated region); HEL (human erythroleukemia); eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); 1 or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); cpm (counts per minute); Ci (Curies); PCR (polymerase chain reaction); ROC (receiver operated characteristics); DSM-IV (Diagnostic and Statistical Manual of Mental Disorders—Fourth Edition); ICD-10 (International Statistical Classification of Diseases and Related Health Problems); ISBRA (International Study for Biomedical Research on Alcoholism); and WHO (World Health Organization).

Equipment and reagents were obtained from the following sources: ABI (Applied Biosystems Inc., Foster City, Calif.); AGTC (Analytical Genetic Testing Center, Inc., Denver, Colo.); Amersham (Amersham Pharmacia Biotec Inc, Piscataway, N.J.); Apple (Apple, Cupertino, Calif.); PE (Perkin-Elmer, Foster City, Calif.) and Pierce (Pierce Biotechnology, Inc., Rockford, Ill.).

### EXAMPLE 1

### Study Subjects and Interviews

Study subjects were recruited for participation in the World Health Organization/International Study for Biomedical Research on Alcoholism (WHO/ISBRA) Collaborative Study on State and Trait Markers for Alcoholism. Subjects were excluded from the study if they manifested medical or psychiatric disorders that made them unable to respond to survey questions or if they used intravenous drugs. Participants from the study centers in Montreal, Helsinki, and Sydney were included in the study and subjects of Caucasian descent were used for association analysis. After the initial screening, and before a translated version of the WHO/ISBRA Interview Schedule was administered, patients provided informed consent. On the same day as the interview, biological samples including urine and blood were collected (Glanz et al., Alcoholism Clin Exp Res, 26:1047-1061, 2002).

The WHO/ISBRA Interview Schedule was adapted from the Alcohol Use and Associated Disabilities Interview Schedule (AUDADIS) developed by the National Institute on Alcohol Abuse and Alcoholism (NIAAA). The Interview Schedule comprised the following major sections: 1) recruitment and setting information; 2) sociodemographic background information; 3) lifetime and 30-day occurrence of medical illness including prescription medicine use; 4) frequency and quantity of beverage-specific alcohol consumption during the prior 30 days; 5) symptoms experienced during ethanol consumption, including treatment; 6) smoking history; 7) history of prescription and illicit drug use; 8) history of depression, antisocial behavior, including treatment for mental illness or emotional problems; and 9) family history of alcohol and drug problems, major depression, and antisocial behavior in first- and second-degree relatives. For use at the various clinical centers, the WHO/ISBRA Interview Schedule was translated from English into five languages: French, Finnish, German, Japanese, and Portuguese.

The WHO/ISBRA interview provides Diagnostic and Statistical Manual of Mental Disorders—Fourth Edition (DSM-IV) and International Statistical Classification of Diseases and Related Health Problems (ICD-10) diagnoses for major depression, alcohol dependence, and dependence on a number of other substances (e.g., sedatives and tranquilizers; heroin, methadone, and other opiates; stimulates and cocaine; cannabis; inhalants; hallucinogens; and anabolic steroids), antisocial personality disorder, and conduct disorder. Medical conditions queried included stomach or duodenal ulcers, hepatomegaly, hepatitis, cirrhosis, kidney disease, pancreatitis, gastritis, thyroid disease, diabetes, hyperlipidemia, tuberculosis, epilepsy, vitamin deficiencies and anemia, emphysema and other lung diseases, arthritis and osteoporosis, hypertension, heart disease, cancer, and immune system problems. The interview data also allowed for medicinal categorization of subjects who were taking medication both in the past month and in the past seven days.

The test-retest Kappa values of the major data elements appearing in the WHO/ISBRA Interview Schedule range from the low of 0.55 for items such as DSM-IV diagnosis of marijuana dependence to values of 1.0 for family history of alcohol dependence in the biological mother.

### EXAMPLE 2

### Blood and DNA Sample Acquisition

Blood was collected at the time of the interview via standard venipuncture technique into vacutainers containing EDTA for preparation of lymphocytes and platelets. Within two hours of collection, the platelets were prepared by centrifuging the blood samples at 700×g for 10 min at room temperature. The platelet-rich plasma layer was transferred to a fresh centrifuge tube and again centrifuged for 10 min at 700×g at room temperature. The upper platelet-rich layer was transferred to a second fresh centrifuge tube and centrifuged at 2800×g for 15 min at room temperature. The platelet pellet was recovered and stored at −70° C. until being shipped on dry ice to the Coordinating Center in Helsinki, Finland, and from there, to Denver, Colo., for analysis. The lymphocyte fraction was prepared by centrifugation and frozen at −70° C. until DNA was extracted at the Assay Center in Denver Colo. Genomic DNA was extracted from the lymphocytes using the Super QUIK-GENE Rapid DNA Isolation Kit, according to the manufacturers' instructions (Analytical Genetic Testing Center).

### EXAMPLE 3

### Platelet Membrane Preparation

The frozen platelet pellet, obtained as described above in Example 2, was thawed and washed at 4° C. For washing, the platelet pellet was suspended in 1.5 ml of 50 mM Tris-HCl (pH 7.5) containing 20 mM EDTA and then centrifuged at 17,000×g for 10 min. This procedure was repeated, and the platelet pellet was then suspended in 1.5 ml of 5 mM Tris-HC 1 (pH 7.5) containing 5 mM EDTA and centrifuged again at 17,000×g for 10 min. The washed platelet pellet was suspended in 1.5 ml of 5 mM Tris-HCl (pH 7.5) containing 1 mM EDTA, using a hand-held Teflon homogenizer. The homogenate was diluted as necessary with 5 mM Tris-HCl (pH 7.5) containing 1 mM EDTA to attain a protein concentration of approximately 200 to 1000 μg/ml and used immediately for the assays of platelet AC activity. Protein determinations were performed using the Bicinchoninic Acid protein microtiter method (Pierce).

### EXAMPLE 4

### Adenylyl Cyclase Assay

Approximately 10 to 50 μg of the platelet membrane protein in 50 μl were added to 200 μl of assay buffer consisting of 25 mM Tris-maleate (pH 7.5), 10 mM theophylline, 5 mM MgCl₂, 0.25 mM ATP, and [α-³²P]ATP (e.g., 1.2 to ×2.0×10⁶ cpm/assay). AC activity was measured in duplicate in assays containing 10 μM forskolin. Following equilibration of the assay mixture at 30° C. for 5 min, the reaction was initiated by adding the platelet membranes. The reaction mixture was then incubated at 30° C. for 10 min. The reaction was terminated by the addition of 750 μl of an ice cold solution containing 4 mM ATP, 1.4 mM cAMP, and 10,000 cpm [³H]cAMP (25 to 40 Ci/mmol), to each assay tube. [³H]cAMP, together with [³²P]cAMP generated by AC, were isolated by sequential chromatography on Dowex and alumina columns (Menninger and Tabakoff, Biol Psychiatry, 42:30-38, 1997), and quantified by liquid scintillation counting.

All reported values were corrected for recovery of [³H]cAMP, and AC activity was expressed as pmol of cAMP generated/mg protein/min. An aliquot of human erythroleukemia (HEL) cell membranes with known levels of AC activity was assayed with each group of samples. The HEL cell membrane preparation was used as a reference standard to control for between-assay variability. The value of the AC activity obtained with HEL cell membranes within each day's assay was divided by the HEL cell membrane activity averaged over the entire project period. The resulting factor was used to standardize all AC activity values obtained on a particular day.

### EXAMPLE 5

### PCR Analysis

Subjects' DNA was genotyped for an [AACA]ₙ microsatellite polymorphism located in the 3′-untranslated region of the AC7 gene by PCR-based methods (Hellevuo et al., Am J Med Genet, 74:95-98, 1997). The AC7 region 2 primer pair used in this analysis yields an approximately 204 bp product from an AC7.R7 allele template (sense 5′-TTC TCC ATG GGT CAA GGA CT-3′ disclosed as SEQ ID NO:3; and antisense 5′-CAT GCA CCA CCT CAA ATC AT-3′ disclosed as SEQ ID NO:4). Shorter PCR products are obtained from AC7 alleles with fewer [AACA] repeats. The present invention is not limited to the use of the above primers, as other oligonucleotides flanking the repeat polymorphism and yielding a PCR product less than 500 bp in length are also suitable. The primers were synthesized using an ABI 394 DNA Synthesizer and the 5′ ends were labeled with ABI's blue fluorescent phosphoramide (6-FAM). All primers were column-purified using ABI's Oligonucleotide Purification Cartridge. PCR was performed on a Perkin-Elmer GeneAmp PCR System 9700 thermocycler. Each reaction contained 50 ng of genomic DNA, 100 ng of each primer, PCR reaction beads (Amersham) and sterile water for a total reaction volume of 25 μl. Cycling conditions were as follows: 94° C. for 12 min, 30 cycles of 94° C. for 20 sec, 55° C. for 1 min, 72° C. for 30 sec, followed by an extension at 72° C. for 1 hr and a 4° C. soak. Positive and negative (no template) controls were included in every set of amplification reactions.

After PCR, aliquots of the samples were mixed with ABI's fluorescent Genescan-500 ROX internal lane standard and electrophoresed on an ABI Prism 310 Genetic Analyzer. Fluorescence data were digitalized and transmitted to a Macintosh G3 computer equipped with Genescan 672 version 3.0 and Genotyper 3.0 software. The PCR product lengths were determined based on internal standards using the Linear Southern Curve options of the analysis software. Representative results are shown as FIG. 3.

### EXAMPLE 6

### Statistical Analysis

Where appropriate, multiple logistic analysis, Pearson's χ² analysis, and odds ratios were used to evaluate the data. Receiver operated characteristics (ROC) curve analysis was used to determine the sensitivity and specificity for AC7.R7 as a marker of phenotype.

Multiple logistic models were constructed to examine variables that contributed significantly to a phenotypic association with the AC7 polymorphisms. The models were constructed by the purposeful selection method (Hosmer and Lemeshow, Applied Logistic Regression, 2nd edition, Wiley:NY, 2000). Before the model building process, statistics and univariate statistical methods (i.e., means, histograms, t tests, x² tests) were implemented to screen the data. Then, all possible univariate logistic regression models with the independent variables were fit. Variables that were significant at the α=0.25 level were included in a saturated model. They were then removed sequentially based on their statistical significance at the α=0.05 level using the log-likelihood ratio test. As they were removed, their potential as confounders was quantified by calculating a change in the coefficients of the models with and without the variable. Correlates that produced changes greater than 15% were considered confounders and were left in the final model. Once the final model of main effects was established, meaningful interaction terms were constructed, and their statistical significance was evaluated with the log-likelihood ratio test. The continuous variables were assessed for linearity in the logit with the fractional polynomial method. Nonlinear terms were either collapsed into meaningful categories or mathematically transformed. The model building process concluded with a series of goodness of fit tests (Hosmer and Lemeshow, supra 2000) and diagnostic statistics (e.g., leverage, Cook's D, deviance, etc.) designed to identify outlying observations and to assess the model's fit and performance. The variables used for this logistic analysis are shown in Table 2.

Unless otherwise stated, the p<0.05 level was used to evaluate the statistical significance of each of the statistical tests. Most analyses were generated using SPSS^{J} for Windows version 9.0 (SPSS, Chicago, Ill.). ROC analysis was performed using Med-Calc^{J} for Windows version 4.30 (Mariakerke, Belgium).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in molecular biology, genetics, or related fields are intended to be within the scope of the following claims.

What is claimed is:

1. A method of identifying individuals predisposed to major depressive disorder comprising:
a) providing a nucleic acid from a human subject; wherein said nucleic acid comprises an adenylyl cyclase type 7 allele;
b) detecting the presence of at least one polymorphism within said adenylyl cyclase type 7 allele; and
c) correlating the presence of said at least one polymorphism with a predisposition to major depressive disorder.

2. The method of claim 1, wherein said at least one polymorphism is a repeat polymorphism.

3. The method of claim 2, wherein said repeat polymorphism is an [AACA]₇ repeat in the 3′ untranslated region of said adenylyl cyclase type 7 allele.

4. The method of claim 1, wherein said subject is Caucasian.

5. The method of claim 1, wherein said subject is female.

6. The method of claim 1, wherein said subject is alcohol-dependent.

7. The method of claim 1, wherein said detecting step is accomplished using at least one technique selected from the group consisting of polymerase chain reaction, heteroduplex analysis, single stand conformational polymorphism analysis, ligase chain reaction, comparative genome hybridization, Southern blotting and sequencing.

8. The method of claim 1, wherein said nucleic acid from said subject is derived from a sample selected from the group consisting of buccal cells, biopsy material and blood.

9. The method of claim 1, further comprising step d) providing a diagnosis to said subject based on the presence or absence of said polymorphism.

10. The method of claim 9, wherein said diagnosis differentiates major depressive disorder from other forms of mental illness.

11. The method of claim 10, wherein said other forms of mental illness comprise bipolar disorder.

12. The method of claim 10, further comprising step e) recommending an antidepressant drug to said subject.

13. A kit for determining if a subject is predisposed to major depressive disorder, comprising:
a) at least one reagent capable of specifically detecting at least one polymorphism in an adenylyl cyclase type 7 allele; and
b) instructions for determining whether a subject is predisposed to major depressive disorder.

14. The kit of claim 13, wherein said at least one polymorphism is a repeat polymorphism.

15. The kit of claim 13, wherein said at least one reagent comprises a nucleic acid probe that hybridizes under stringent conditions to a nucleic acid sequence selected from the group consisting of the coding strand of the adenylyl cyclase type 7 gene, and the noncoding strand of the adenylyl cyclase type 7 gene.

16. The kit of claim 13, wherein said at least one reagent comprises a sense primer and an antisense primer flanking said at least one polymorphism in said adenylyl cyclase type 7 allele.

17. The kit of claim 16, wherein at least one of said primers comprises a fluorescent tag.

18. The kit of claim 13, wherein said instructions comprise instructions required by the United States Food and Drug Administration for use in in vitro diagnostic products.

19. The kit of claim 13, further comprising at least one reagent capable of specifically detecting at least one polymorphism in an additional allele associated with major depressive disorder.

20. A method of screening compounds, comprising:
a) providing:
i) at least one cell comprising an adenylyl cyclase type 7 allele with a tetranucleotide repeat polymorphism, and
ii) one or more test compounds; and
b) contacting said at least one cell with said test compound; and
c) detecting a change in adenylyl cyclase type 7 in said at least one cell in the presence of said test compound relative to the absence of said test compound.

21. The method of claim 20, wherein said detecting comprises detecting a change in adenylyl cyclase type 7 mRNA.

22. The method of claim 20, wherein said detecting comprises detecting a change in a change in adenylyl cyclase type 7 polypeptide.

23. The method of claim 20, wherein said detecting comprises detecting a change in adenylyl cyclase type 7 enzymatic activity.

24. The method of claim 20, wherein said cell is a platelet.

25. The method of claim 20, wherein said test compound comprises a drug.

26. A method of identifying individuals predisposed to major depressive disorder, comprising:
a) providing a nucleic acid sample from a subject, said sample containing an adenylyl cyclase type 7 allele;
b) correlating the identity of said adenylyl cyclase type 7 allele with a predisposition to major depressive disorder.

27. The method of claim 26, wherein said identity of said adenylyl cyclase type 7 allele is accomplished using at least one technique selected from the group consisting of polymerase chain reaction, heteroduplex analysis, single stand conformational polymorphism analysis, ligase chain reaction, comparative genome hybridisation, Southern blotting and sequencing.

28. The method of claim 26, wherein said nucleic acid sample from said subject is selected from the group consisting of buccal cells, biopsy material and blood.

29. The method of claim 26, further comprising step c) providing a diagnosis to said subject based on the identity of said adenylyl cyclase type 7 allele.

30. The method of claim 29, wherein said diagnosis differentiates major depressive disorder from other forms of mental illness.

31. The method of claim 30, wherein said other forms of mental illness comprise bipolar disorder.

32. The method of claim 30, further comprising step d) recommending an antidepressant drug to said subject.


SEQUENCE LISTING (4 sequences)

SEQ ID NO: 1 - DNA, 6196 bp, Homo sapiens
tgaggaactgcgtgtggagtcagcccagtctggatgcacaggaggatgctggcggcacagtgagtgaggcctggtgccagagctgtgcggaccccttgttggccatggagcagcaggcccagaggccctctccccagccctgcttgcctgcctcggagaggacagaggcctaggcccacgggggagggtgttggcagacagatgccctccaggccctggggcctccttaacggccccttaacgacacgcgtgccaagggtggaggatgccagccaaggggcgctacttcctcaacgagggcgaggagggccctgaccaagatgcgctctacgagaagtaccagctcaccagccagcatgggccgctgctgctcacgctcctgctggtggccgccactgcctgcgtggccctcatcatcattgccttcagccagggggacccctccagacaccaggccattctgggcatggcgttcctggtgctggcggtgtttgcggccctctctgtgctgatgtacgtcgagtgtctcctgcggcgctggctcagggccttggcgctgctcacctgggcctgcttggtggcgctgggctatgtgctggtgttcgacgcatggacaaaggcggcctgtgcgtgggagcaggtgcccttcttcctgttcattgtcttcgtggtgtacacactactgcccttcagcatgcggggcgctgtcgccgttggggccgtctccactgcctcccacctcctggtgctcggttctttgatgggaggcttcacgacacccagtgtccgggtggggctgcagctgctggccaacgcagtcatcttcctgtgtgggaacctgacaggcgccttccacaagcaccaaatgcaggatgcgtcccgggacctcttcacctacactgtgaagtgcatccagatccgccggaagctgcgcatcgagaagcgccagcaggagaacctgctgctgtcagtgcttccggcccacatctccatgggcatgaagctggccatcatcgaacggctcaaggagcatggtgaccgtcgctgcatgcctgacaacaacttccacagcctctacgtcaagaggcaccagaatgtcagcatcctctatgcggacatcgtgggcttcacgcagctggccagcgactgttctcccaaggagctggtggtggtgctgaatgagctctttggcaagttcgaccagatcgccaaggccaacgagtgcatgcgaatcaagatcctcggcgactgctactactgtgtatcgggcctgcccgtgtcgctgcctacccacgcccggaactgcgtgaagatggggctggacatgtgccaggccatcaagcaggtgcgggaggccacgggcgtggacatcaacatgcgtgtgggcatacactcggggaatgtgctgtgcggggtcatcgggctgcgcaagtggcagtatgacgtgtggtcccacgacgtgtccctggccaaccggatggaggcagccggagtacccggccgggtgcacatcacggaggccacgctaaagcacctggacaaggcgtacgaggtggaggatgggcacgggcagcagcgggacccctacctcaaggagatgaacatccgcacctacctggtcatcgacccccggagccagcagccacccccgcccagccaacacctccccaggcccaagggggacgcggccctgaagatgcgggcgtcagtgcgcatgacccggtacctcgagtcctggggggcggcacggccctttgcacatctcaaccaccgtgagagcgtgagcagtggtgagacccacgtccccaacgggcggaggcctaagagcgttccccagcgccaccgccggaccccagacagaagcatgtcccccaaggggcggtcggaggatgactcgtacgatgacgagatgctgtcagccattgaggggctcagctccacgaggccctgctgctccaagtccgatgacttctacacctttgggtccatcttcctggagaagggctttgagcgcgagtaccgcctggcacccatcccccgggcccgccacgactttgcctgcgccagcctgatcttcgtctgcatcctgctcgtccatgtcctgctcatgcccaggacggcggcactgggtgtgtccttcgggctggtggcctgtgtactggggctggtgctgggcctgtgctttgccaccaagttctcgaggtgctgcccagctcgggggacgctctgcactatctctgagagggtggagacacagcccctgctgaggctgaccctggccgtcctgaccatcggcagcctgctcactgtggccatcatcaacctgcccctgatgcctttccaagttccagagctgcctgttggcaatgagacaggcctactggccgcgagcagcaagacaagagccctgtgtgagcccctcccgtactacacctgcagctgtgtcctgggcttcatcgcctgctcggtcttcctgaggatgagcctggagccaaaggttgtgctgctgacagtggccctggtggcctacctggtgctcttcaacctctccccatgctggcagtgggactgctgcggccaaggcctgggcaacctcaccaagcccaacggcaccaccagtggcacccctagctgttcctggaaggacctgaagaccatgaccaatttctacctggtcctgttctacatcaccctgcttacactctccagacagattgactattactgccgcttggactgcctatggaagaagaagttcaagaaggagcacgaggagtttgagaccatggagaacgtgaaccgccttcttctggagaacgtcctgccagcccacgtggctgcccactttatcggtgacaagttaaacgaggactggtaccatcagtcctatgactgcgtctgtgtcatgtttgcctccgtgccggacttcaaagtgttctacacagagtgcgatgtcaacaaagaagggctggagtgcctacgcctgctcaatgagatcattgccgacttcgacgagctcctactgaagcccaagttcagcggcgtggagaagatcaagaccatcggcagcacgtacatggcagctgcagggctcagcgtcgcctcagggcacgagaaccaggagctggagcggcagcatgcccacattggtgtcatggtggagttcagcatcgccctgatgagtaagctggacggcatcaacaggcactccttcaactccttccgcctccgcgtcggcataaaccatgggcctgtgattgctggagtgattggggcccgaaaacctcagtatgacatctggggaaacactgtcaatgtggccagccgaatggaaagcactggagaacttgggaaaatccaggttaccgaggagacctgcaccatcctccagggcctcgggtactcttgtgaatgccgtggcctgatcaacgtcaaaggcaaaggcgagctgaggacttactttgtctgtacggacactgccaagtttcaggggctggggctgaactgagggctcctgctggattccgaaaaggccgggaagccagtctccttccctgaagcaagcccaggagaagactctccgccccacgccaatcccaaaggcatgcagatggctgtgcatgttggcttctttggacctgcactggaggatttctcagacacatgcaccagattctggctcgaagcagccactgagccataatgcgcaggggaggccagaagctctgtgcctggtctgtaacagtttccaggccagctggagaatgttcactggttcggggctgactttgagatctttgttccctgaggtgccaggcaggcaactttagcacatgatgaaaacagacttccacctcagtggcctgtgggcacgcacaagtgaggtctgtttttctagacaccaagggggagtaagctgagctgtctagcacggattggagactccctctccctggtgggcctggcaatgacagcatttctcacagaggcattctggtaaatgaagctgaaaggggtgttttacatctgtaaacggtttcaaacaggtagagagaaaaacaccacaattaacactgttactttttgccttgtctggcatgtttgttttaaatgaatacattaatggggtttttatccttttgaatgacttttcagacactagacataaatctcttccctccagtgtatgctctgcctttttaaccactgacatgtaaggaggactactgtctagcatcagcttatggggtcagctggctgtggggatagagtcctgaggaatgtggtcacagcaagaaggcggggagcagcagagccttgcctttgaatgaggcagcttgtgaggcaagcattctggagagaggtgctttgaaagtaaggtgcggcctttcacctcttccttgattactcacacatctttgcgttctcccctgccgtccttcaactgtatcttacttttcttaccagaaaggaatggagtctgtttagagacaacttggacaacctgtgagtgcatctcttctttcctttagtcttcacagctaactctggagagcttcaaaactagaaggatctactccgcatgggtgcatgcagaggctcctggatctgggaagcccgccccctcacaaatgctgagccgttcttgctctgaaactgcgtgagtcaaggcaaatgcaaaaagccaggttttggggatgtgtcttactgtgcttcaacttcccaaggaattgaaagtcaacctaactgtaacaacagggtgagaaatgaccaaactgcccgtgactttttctgaatggacttcataaccggaagacttaaccggtggcctcatcaccagagcatcgccaggatttctaatgcactcagtttccctacatagcagggattcttagctaggtgtccccatgaaccccgtaaagttctacacaaagtcttgcatacaggagcctttacaagatgattatacagggttgcagattgggtgactgaccagacttgttggggtcctgggatgagttgccccgggctgcaaattaagagtacagctaagtgcgggggtggcggtggagggaacgaaaattgaacctgtctgcctgtgctgtgtcgtgtggctttatcagcccgaggaagggcaggtgtattctaatttgcacaaaggtgctgggtagactagtggcagctctcatgtgctgcacataagtggaatcagtatgaatagaagaacttgctgtataaaggaatttcatggcaacaatgctggtaagggcaattagcctcgcttaagttgccttttttacacaccaaaactttttacatgaagggctggtttcacatgaatactatactgaaatctgtgccacaccaaaactttttacatgaagggctggtttcacatgaatactatactgaaatctgtgctctcaagatctagcagtgaccagggctgcccggcgggggctctcctggcaagtcaggaaggtttctgttgctaatataacatagaaacacattagtgcactgggcctctctgaggtcagcatatttgtactcttggaatatttgtttttttcttcagtaacaacagaaaccccagttgggagtttaacaaataactgactaccactcactcatgcatttttatttccaattaaagcaaagcactgtgctgtgctcagataataatagtttgtaagtaaaagtttttagttttcagtgttcaggttatagaatataactgaccataaaaattacctgcaggtattttctttttatgaacttgtttttaaattaccaagtaattactggtgtcattttgttttatgacagacacacgtatctaacaaacaaacaaacagtgaccttctccatgggtcaaggacttccttacaatttctcctgagttaacttttgtgaaaataatacctaaggttttctggcttattgaggaaatttcctaacaaacaaacaaacaaacaaacagaagagaagatcattaaccactgtatactttgtgtatataataggtcagtgtaaagaaatatgatttgaggtggtgcatgcaagtaactagggtttattctatataatgaatatttatagatctgtaacatttgtttcaaaatgctgtttcatttttataaagtaccagtgtttagctgctttttatacattaaattagcaatttgaaaaactc

SEQ ID NO: 2 - DNA, 28 bp, Homo sapiens
aacaaacaaacaaacaaacaaacaaaca

SEQ ID NO: 3 - DNA, 20 bp, Artificial Sequence
  Synthetic
ttctccatgggtcaaggact

SEQ ID NO: 4 - DNA, 20 bp, Artificial Sequence
  Synthetic
catgcaccacctcaaatcat